SEQUENCE LISTING (60 sequences)

SEQ ID NO: 1 - DNA, 31 bp, Artificial Sequence
  Synthetic oligonucleotide
aggcaatctttctcaatctattgaagagttt

SEQ ID NO: 2 - DNA, 28 bp, Artificial Sequence
  Synthetic oligonucleotide
attcacacagatggttaagattcttgga

SEQ ID NO: 3 - DNA, 32 bp, Artificial Sequence
  Synthetic oligonucleotide
tggaggcctaactaatttacaatatctcttct

SEQ ID NO: 4 - DNA, 23 bp, Artificial Sequence
  Synthetic oligonucleotide
tcaaatggcatggatacccgttt

SEQ ID NO: 5 - DNA, 28 bp, Artificial Sequence
  Synthetic oligonucleotide
tgggattgtgtgaagcattttagtaagt

SEQ ID NO: 6 - DNA, 24 bp, Artificial Sequence
  Synthetic oligonucleotide
ggcttcttctggatgtcctgtaaa

SEQ ID NO: 7 - DNA, 20 bp, Artificial Sequence
  Synthetic oligonucleotide
cccaacgaagaggacgtctt

SEQ ID NO: 8 - DNA, 24 bp, Artificial Sequence
  Synthetic oligonucleotide
gcactccccaaagacctaattagc

SEQ ID NO: 9 - DNA, 22 bp, Artificial Sequence
  Synthetic oligonucleotide
tcgaacacacgaccttgtatgg

SEQ ID NO: 10 - DNA, 25 bp, Artificial Sequence
  Synthetic oligonucleotide
ttgagggaactccagttactaagga

SEQ ID NO: 11 - DNA, 24 bp, Artificial Sequence
  Synthetic oligonucleotide
gctgcatcaggcaaatggtttata

SEQ ID NO: 12 - DNA, 27 bp, Artificial Sequence
  Synthetic oligonucleotide
accaggtgcatacaaaattgttcattg

SEQ ID NO: 13 - DNA, 23 bp, Artificial Sequence
  Synthetic oligonucleotide
ctatcttgtcctagacgctgcaa

SEQ ID NO: 14 - DNA, 26 bp, Artificial Sequence
  Synthetic oligonucleotide
caagagtttcttctcttcagcagtgt

SEQ ID NO: 15 - DNA, 24 bp, Artificial Sequence
  Synthetic oligonucleotide
cacatgggtctgagtgataaggat

SEQ ID NO: 16 - DNA, 27 bp, Artificial Sequence
  Synthetic oligonucleotide
cagctctattttggactgtatgaggta

SEQ ID NO: 17 - DNA, 21 bp, Artificial Sequence
  Synthetic oligonucleotide
ggcaactggcttgaagaacat

SEQ ID NO: 18 - DNA, 23 bp, Artificial Sequence
  Synthetic oligonucleotide
ccgaggtccaattcaactcactt

SEQ ID NO: 19 - DNA, 24 bp, Artificial Sequence
  Synthetic oligonucleotide
caatatccatgcaatccgagcaaa

SEQ ID NO: 20 - DNA, 23 bp, Artificial Sequence
  Synthetic oligonucleotide
cggtggaattgaatgtggctaag

SEQ ID NO: 21 - DNA, 25 bp, Artificial Sequence
  Synthetic oligonucleotide
tggtcaagttgctgatctctttagg

SEQ ID NO: 22 - DNA, 35 bp, Artificial Sequence
  Synthetic oligonucleotide
cattcattgaaatgttgaaggatattaaacttggt

SEQ ID NO: 23 - DNA, 30 bp, Artificial Sequence
  Synthetic oligonucleotide
ggatttcaagcttatcaaatcaccaaatga

SEQ ID NO: 24 - DNA, 26 bp, Artificial Sequence
  Synthetic oligonucleotide
atgcgactattacacaggttcaactt

SEQ ID NO: 25 - DNA, 24 bp, Artificial Sequence
  Synthetic oligonucleotide
cgtgggatttggaattttggcaat

SEQ ID NO: 26 - DNA, 25 bp, Artificial Sequence
  Synthetic oligonucleotide
tgttctccttcataagttgcaacga

SEQ ID NO: 27 - DNA, 25 bp, Artificial Sequence
  Synthetic oligonucleotide
ctgggagtgggattatgattatgct

SEQ ID NO: 28 - DNA, 20 bp, Artificial Sequence
  Synthetic oligonucleotide
tcaagccatgtggagcactt

SEQ ID NO: 29 - DNA, 25 bp, Artificial Sequence
  Synthetic oligonucleotide
ggtctcaacgtgttatctggagtta

SEQ ID NO: 30 - DNA, 25 bp, Artificial Sequence
  Synthetic oligonucleotide
ccctaatcaccttccctttcttgtt

SEQ ID NO: 31 - DNA, 31 bp, Artificial Sequence
  Synthetic oligonucleotide
tccaattttctcaagcttaaaccaatttagc

SEQ ID NO: 32 - DNA, 21 bp, Artificial Sequence
  Synthetic oligonucleotide
cgccatcctcaaacgaaaagc

SEQ ID NO: 33 - DNA, 20 bp, Artificial Sequence
  Synthetic oligonucleotide
acgtcgaacgccatccttag

SEQ ID NO: 34 - DNA, 25 bp, Artificial Sequence
  Synthetic oligonucleotide
tgctttcctaatgaagcgttatcgt

SEQ ID NO: 35 - DNA, 21 bp, Artificial Sequence
  Synthetic oligonucleotide
cttgtggcacctacccaatgt

SEQ ID NO: 36 - DNA, 25 bp, Artificial Sequence
  Synthetic oligonucleotide
acttctctgatgaatgcatggtgaa

SEQ ID NO: 37 - DNA, 21 bp, Artificial Sequence
  Synthetic oligonucleotide
agcaacacgtccatgaagtca

SEQ ID NO: 38 - DNA, 25 bp, Artificial Sequence
  Synthetic oligonucleotide
gcaagtaggtctggattgttcaaca

SEQ ID NO: 39 - DNA, 25 bp, Artificial Sequence
  Synthetic oligonucleotide
caggtccgtagttgaaattccaaga

SEQ ID NO: 40 - DNA, 25 bp, Artificial Sequence
  Synthetic oligonucleotide
ggtaaagaacccaatacggttacgt

SEQ ID NO: 41 - DNA, 16 bp, Artificial Sequence
  Synthetic oligonucleotide
aatgccgccactaatg

SEQ ID NO: 42 - DNA, 16 bp, Artificial Sequence
  Synthetic oligonucleotide
tccaaggccacattcc

SEQ ID NO: 43 - DNA, 16 bp, Artificial Sequence
  Synthetic oligonucleotide
caaggctcgattcctg

SEQ ID NO: 44 - DNA, 16 bp, Artificial Sequence
  Synthetic oligonucleotide
tttgcccgtgagtttc

SEQ ID NO: 45 - DNA, 16 bp, Artificial Sequence
  Synthetic oligonucleotide
ctgcctgccataagct

SEQ ID NO: 46 - DNA, 16 bp, Artificial Sequence
  Synthetic oligonucleotide
cctccatgcatctttg

SEQ ID NO: 47 - DNA, 15 bp, Artificial Sequence
  Synthetic oligonucleotide
ctgccgcccatgctt

SEQ ID NO: 48 - DNA, 14 bp, Artificial Sequence
  Synthetic oligonucleotide
caacagccaatccc

SEQ ID NO: 49 - DNA, 16 bp, Artificial Sequence
  Synthetic oligonucleotide
cccgcactgtattctt

SEQ ID NO: 50 - DNA, 16 bp, Artificial Sequence
  Synthetic oligonucleotide
acccaaacacgaaccc

SEQ ID NO: 51 - DNA, 16 bp, Artificial Sequence
  Synthetic oligonucleotide
ctggagctgaaacttt

SEQ ID NO: 52 - DNA, 15 bp, Artificial Sequence
  Synthetic oligonucleotide
tcttgcgtcaaactc

SEQ ID NO: 53 - DNA, 16 bp, Artificial Sequence
  Synthetic oligonucleotide
ttgaaacgccaatttt

SEQ ID NO: 54 - DNA, 16 bp, Artificial Sequence
  Synthetic oligonucleotide
ccacaacgaatctttg

SEQ ID NO: 55 - DNA, 16 bp, Artificial Sequence
  Synthetic oligonucleotide
atgaccgccggataaa

SEQ ID NO: 56 - DNA, 16 bp, Artificial Sequence
  Synthetic oligonucleotide
ctcccttccactttcc

SEQ ID NO: 57 - DNA, 16 bp, Artificial Sequence
  Synthetic oligonucleotide
tcaccctgtaattttc

SEQ ID NO: 58 - DNA, 15 bp, Artificial Sequence
  Synthetic oligonucleotide
ccccaaggcttctcc

SEQ ID NO: 59 - DNA, 16 bp, Artificial Sequence
  Synthetic oligonucleotide
cccggccatagtaacc

SEQ ID NO: 60 - DNA, 15 bp, Artificial Sequence
  Synthetic oligonucleotide
cagcggcattcccac

(19) United States
(12) Patent Application Publication
(10) Pub. No.: US 2017/0145478 A1
(43) Pub. Date: May 25, 2017

(54) BIOMARKERS FOR THE PRE-SYMPTOMATIC DIAGNOSIS OF HUANGLONGBING (HLB) IN CITRUS AND USE THEREOF

(71) Applicants: Los Alamos National Security, LLC, Los Alamos, NM (US); The United States of America, as represented by the Secretary of Agriculture, Washington, DC (US)

(72) Inventors: Goutam Gupta, Los Alamos, NM (US); Eddie Wayne Stover, Fort Pierce, FL (US); Paige Pardington, Los Alamos, NM (US); Melinda Wren, Los Alamos, NM (US)

(73) Assignees: Los Alamos National Security, LLC, Los Alamos, NM (US); The United States of America, as represented by the Secretary of Agriculture, Washington, DC (US)

(21) Appl. No.: 15/360,633

(22) Filed: Nov. 23, 2016

Related U.S. Application Data
(60) Provisional application No. 62/258,831, filed on Nov. 23, 2015.

(51) Int. Cl. C12Q 1/68

(52) U.S. Cl. CPC C12Q 1/689; C12Q 2600/158; C12Q 2600/16; C12Q 2600/156

(57) ABSTRACT

The identification of genes upregulated following infection of citrus trees by Liberibacter, the causative agent of huanglongbing (HLB), is described. Methods for detecting pre-symptomatic HLB in citrus trees by detecting expression of one or more genes overexpressed following infection by Liberibacter is also described.

### CROSS REFERENCE TO RELATED APPLICATIONS

This application claims the benefit of U.S. Provisional Application No. 62/258,831, filed Nov. 23, 2015, which is herein incorporated by reference in its entirety.

### ACKNOWLEDGMENT OF GOVERNMENT SUPPORT

This invention was made with government support under Contract No. DE-AC52-06NA25396 awarded by the U.S. Department of Energy, and under Project No. 6618-21000-014-00D awarded by the Agricultural Research Service of the U.S. Department of Agriculture. The government has certain rights in the invention.

### FIELD

This disclosure concerns the identification and use of pre-symptomatic biomarkers indicative of huanglongbing (HLB) in citrus trees, a disease that is caused by infection with the gram-negative bacterium Candidatus Liberibacter.

### BACKGROUND

Huanglongbing (HLB) is the most devastating and economically damaging disease of citrus (Grafton-Cardwell et al., Annu Rev Entomol 58:413-432, 2013; Brlansky et al., Huanglongbing (Citrus Greening), Publication SP-43, 2007 Florida Citrus Pest Management Guide, Florida Cooperative Extension Service, Institute of Food and Agricultural Sciences, University of Florida). Loss of productivity and eventual death of the trees pose a great threat to the citrus industries in the U.S. and other citrus producing countries. All citrus cultivars are susceptible to HLB, although the disease severity varies among different cultivars (Cevallos-Cevallos et al., Plant Physiol Biochem 53:69-76, 2012).

HLB was first discovered in Florida in 2005 and is now widespread across the state. It is estimated that greater than 70% of all trees are already infected in Florida. HLB is also a threat for California, Arizona and Texas. Judging by the trend in other countries, HLB will continue to spread within the U.S. commercial citrus industry, causing severe decline in production and significant economic loss. This has been the case in Florida, where there is a 10-20% per year decline in production, decrease in fruit quality and increase in production costs (National Agriculture Statistics Service: Citrus Fruits 2013 Summary), which has already forced some growers into bankruptcy. In view of this, it is clear that the 20 billion dollar U.S. citrus industry faces a serious threat from HLB. HLB-resistant citrus is the long-term protection strategy, which may be developed using consumer acceptable genetic engineering steps. However, short-term strategies are needed for the treatment of citrus trees already infected with the HLB-causing Candidatus Liberibacter.

HLB is a vector-borne disease transmitted by the Asian citrus psyllid (ACP). While these psyllids are abundant in southern California and are now established in Arizona, so far only a few HLB-infected trees have been documented in California. Psyllid control and Liberibacter surveillance are coordinated efforts in California and Arizona to minimize insect spread and prevent establishment of HLB, making early detection of non-symptomatic trees of utmost importance.

In Florida, aggressive psyllid control is implemented by most individual growers to minimize spread to remaining healthy trees, but it has proven impossible to eliminate HLB spread by psyllid control. Currently, the presence of HLB infection is determined by monitoring for distinctive symptoms, and verified by PCR for Liberibacter in tree samples. However, it may take several years for the HLB symptoms to appear after the initial Liberibacter exposure. The initial distribution of Liberibacter in the tree is not uniform so PCR scanning of non-symptomatic trees has not been effective, with many false negatives. Therefore, infected but non-symptomatic trees remain undiagnosed and continue to be dangerous inoculum sources for disease spread. Thus, a robust platform for early diagnosis of HLB is urgently needed.

### SUMMARY

Disclosed herein is the identification of genes exhibiting altered expression in pre-symptomatic citrus trees following infection by Liberibacter, the causative agent of HLB. Further disclosed are methods for detecting pre-symptomatic infection by Liberibacter in a citrus plant by evaluating expression of at least one of the identified genes.

Provided herein is method of detecting pre-symptomatic infection by Candidatus Liberibacter in a citrus plant. In some embodiments, the method includes measuring expression of at least one, at least two or at least three biomarker genes in a leaf sample obtained from the citrus plant, and detecting pre-symptomatic infection by Candidatus Liberibacter in the citrus plant if expression of the gene(s) is increased compared to a control. In some examples, the at least one, at least two or at least three genes are selected from the orange1.1t04419.1, Cs9g12160.1, Cs2g08750.1, orange1.1t03694.1, orange1.1t04702.1, Cs7g06330.1, Cs5g33540.1, orange1.1t04376.1, orange1.1t03769.1, Cs9g15430.1, Cs5g16850.1, Cs5g16920.1, Cs5g16770.1, Cs5g16780.1, Cs6g04140.1, Cs2g10910.1, Cs5g27580.1, Cs8g01850.1, Cs5g21900.1 and Cs8g01840.1 genes of Citrus sinensis, or a homolog thereof in another citrus plant species.

Also provided herein is a kit for detecting pre-symptomatic infection by Candidatus Liberibacter in a citrus plant. In some embodiments, the kit comprises at least one primer pair listed in Table 2 and/or at least one probe listed in Table 3.

The foregoing and other objects, features, and advantages of the invention will become more apparent from the following detailed description, which proceeds with reference to the accompanying figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic representation of the pathogen-associated molecular pattern (PAMP)-triggered, effector-triggered and plant hormone (salicylic acid—SA, jasmonic acid—JA, and ethylene—ET) pathways induced early upon pathogen exposure. These pathways are also affected by the reactive oxygen species and calcium release generated by pathogen attack.

FIG. 2 is a schematic of the greenhouse study design described in Example 1. A cage was attached at the end of a branch of each tree. The cage contained either Liberibacter (+) or Liberibacter (−) psyllids. Each tree was monitored for 0-24 weeks. The trees were divided into infected and uninfected groups and were monitored for 0, 2, 4, 8, 12 and 24 weeks post-inoculation. For each time-point, RNA from leaf samples at 15, 30 and 60 cm from the site of inoculation were collected.

FIG. 3 is a schematic of the five coupled citrus pathways induced upon early Liberibacter infection. Differential gene expression analysis of 44,000 citrus genes revealed that genes belonging to effector-triggered immunity (ETI), PAMP-triggered immunity (PTI), SA, JA and ET signaling pathways were significantly altered. The final output of these pathways are the induction of citrus immune defense and pathogenesis related genes. Expression levels of multiple genes belonging to these pathways define early infection stages and disease progression.

FIG. 4 shows that of the 80 discovered genes, 20 genes (left most panel) show similar expression patterns for most of the post-inoculation times and at the three sampled distances. The same amount of total RNA was used for each sample. The shaded boxes indicate the data collection for the different samples (each time post-inoculation at each sample distance). The top four listed genes represent the citrus receptor family, the next three listed genes represent the transcription factor family, and the remaining genes represent the defense and pathogenesis-related gene family.

FIGS. 5A-5B are tables showing citrus genes overexpressed following Liberibacter exposure. FIG. 5A lists genes identified as overexpressed two weeks and four weeks post-inoculation at the three sample distances. FIG. 5B lists genes identified as overexpressed eight weeks and twenty-four weeks post-inoculation at the three sample distances.

### SEQUENCE LISTING

The nucleic acid sequences listed in the accompanying sequence listing are shown using standard letter abbreviations for nucleotide bases, as defined in 37 C.F.R. 1.822. Only one strand of each nucleic acid sequence is shown, but the complementary strand is understood as included by any reference to the displayed strand. The Sequence Listing is submitted as an ASCII text file, created on Nov. 9, 2016, 11.9 KB, which is incorporated by reference herein. In the accompanying sequence listing:

SEQ ID NOs: 1-40 are nucleic acid primer sequences for amplification of citrus genes.

SEQ ID NOs: 41-60 are nucleic acid probe sequences.

### DETAILED DESCRIPTION

I. Abbreviations

ACP Asian citrus psyllid

ET ethylene

ETI effector-triggered immunity

HLB huanglongbing

JA jasmonic acid

LRR leucine-rich repeat

PAMP pathogen-associated molecular pattern

PRR PAMP recognition receptors

PTI PAMP-triggered immunity

qPCR quantitative polymerase chain reaction

SA salicylic acid

II. Terms and Methods

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Array: An arrangement of molecules, particularly biological macromolecules (such as polypeptides or nucleic acids) or biological samples (such as tissue sections) in addressable locations on a substrate, usually a flat substrate such as a membrane, plate or slide. The array may be regular (arranged in uniform rows and columns, for instance) or irregular. The number of addressable locations on the array can vary, for example from a few (such as three) to more than 50, 100, 200, 500, 1000, 10,000, or more. A “microarray” is an array that is miniaturized to such an extent that it benefits from microscopic examination for evaluation.

Within an array, each arrayed molecule (e.g., oligonucleotide) or sample (more generally, a “feature” of the array) is addressable, in that its location can be reliably and consistently determined within the at least two dimensions on the array surface. Thus, in ordered arrays the location of each feature is usually assigned to a sample at the time when it is spotted onto or otherwise applied to the array surface, and a key may be provided in order to correlate each location with the appropriate feature.

Often, ordered arrays are arranged in a symmetrical grid pattern, but samples could be arranged in other patterns (e.g., in radially distributed lines, spiral lines, or ordered clusters). Arrays are computer readable, in that a computer can be programmed to correlate a particular address on the array with information (such as identification of the arrayed sample and hybridization or binding data, including for instance signal intensity). In some examples of computer readable array formats, the individual spots on the array surface will be arranged regularly, for instance in a Cartesian grid pattern, that can be correlated to address information by a computer.

The sample application spot (or feature) on an array may assume many different shapes. Thus, though the term “spot” is used herein, it refers generally to a localized deposit of nucleic acid or other biomolecule, and is not limited to a round or substantially round region. For instance, substantially square regions of application can be used with arrays, as can be regions that are substantially rectangular (such as a slot blot-type application), or triangular, oval, irregular, and so forth. The shape of the array substrate itself is also immaterial, though it is usually substantially flat and may be rectangular or square in general shape.

Candidatus Liberibacter: A genus of gram-negative bacteria in the Rhizobiaceae family. Members of this genus are primarily plant pathogens transmitted by psyllids. Candidatus Liberibacter asiaticus: A species of the genus Candidatus Liberibacter that is the causative agent of huanglongbing. Candidatus Liberibacter asiaticus originated in Asia and is transmitted by the Asian citrus psyllid Diaphorina citri. Candidatus Liberibacter asiaticus is also known as “Liberibacter asiaticus.”

Citrus plant: In the context of the present disclosure, a citrus plant includes any cultivated genotype in the genus Citrus, such as orange, mandarin, lemon, lime or grapefruit plants.

Control: A reference standard, for example a positive control or negative control. A positive control is known to provide a positive test result. A negative control is known to provide a negative test result. However, the reference standard can be a theoretical or computed result, for example a result obtained in a population. In some embodiments herein, the level of biomarker expression in a leaf sample is compared to a control sample, such as an uninfected leaf sample, a historical value or a standard value.

Diaphorina citri: A sap-sucking hemipteran insect in the family Psyllidae. This insect is an important pest of citrus as it transmits the bacteria responsible for HLB.

Fluorophore: A chemical compound, which when excited by exposure to a particular wavelength of light, emits light (i.e., fluoresces), for example at a different wavelength. Fluorophores can be described in terms of their emission profile, or “color.” Green fluorophores, for example Cy3, FITC, and Oregon Green, are characterized by their emission at wavelengths generally in the range of 515-540 λ. Red fluorophores, for example Texas Red, Cy5 and tetramethylrhodamine, are characterized by their emission at wavelengths generally in the range of 590-690 λ.

Examples of fluorophores are provided in U.S. Pat. No. 5,866,366 to Nazarenko et al., and include for instance: 4-acetamido-4′-isothiocyanatostilbene-2,2′disulfonic acid, acridine and derivatives such as acridine and acridine isothiocyanate, 5-(2′-aminoethyl)aminonaphthalene-1-sulfonic acid (EDANS), 4-amino-N-[3-vinylsulfonyl)phenyl]naphthalimide-3,5 disulfonate (Lucifer Yellow VS), N-(4-anilino-1-naphthyl)maleimide, anthranilamide, Brilliant Yellow, coumarin and derivatives such as coumarin, 7-amino-4-methylcoumarin (AMC, Coumarin 120), 7-amino-4-trifluoromethylcouluarin (Coumaran 151); cyanosine; 4′,6-diaminidino-2-phenylindole (DAPI); 5′, 5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as cosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein, fluorescein isothiocyanate (FITC), and QFITC (XRITC); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron .RTM. Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives.

Other contemplated fluorophores include GFP (green fluorescent protein), Lissamine™, diethylaminocoumarin, fluorescein chlorotriazinyl, naphthofluorescein, 4,7-dichlororhodamine and xanthene and derivatives thereof. Other fluorophores known to those skilled in the art may also be used.

Examples of fluorophores that are sensitive to ion concentration (such as Ca²⁺ concentration or flux) include, but are not limited to, bis-(1,3-dibutylbarbituric acid)trimethine oxonol (DiBAC4(3) (B-438), Quin-2 (AM Q-1288), Fura-2 (AM F-1225), Indo-1 (AM I-1226), Fura-3 (AM F-1228), Fluo-3 (AM F-1241), Rhod-2, (AM R-1244), BAPTA (AM B-1205), 5,5′-dimethyl BAPTA (AM D-1207), 4,4′-difluoro BAPTA (AM D-1216), 5,5′-difluoro BAPTA (AM D-1209), 5,5′-dibromo BAPTA (AM D-1213), Calcium Green (C-3011), Calcium Orange (C-3014), Calcium Crimson (C-3017), Fura-5 (F-3023), Fura-Red (F-3020), SBFI (S-1262), PBFI (P-1265), Mag-Fura-2 (AM M-1291), Mag-Indo-1 (AM M-1294), Mag-Quin-2 (AM M-1299), Mag-Quin-1 (AM M-1297), SPQ (M-440), SPA (S-460), Calcien (Fluorescein-bis(methyliminodiacetic acid); Fluorexon), and Quin-2 (2-{[2-Bis-(carboxymethyl)amino-5-methylphenoxy]-methyl}-6-methoxy-8-bis-(carboxymethyl)aminoquinoline tetrapotassium salt).

Huanglongbing (HLB): A disease of citrus caused by the vector-transmitted pathogen Candidatus Liberibacter asiaticus. HLB is also known as “citrus greening disease.” HLB is distinguished by the common symptoms of yellowing of the veins and adjacent tissues, followed by splotchy mottling of the entire leaf, premature defoliation, dieback of twigs, decay of feeder rootlets and lateral roots, and decline in vigor, frequently followed by the death of the entire plant. Severely affected trees have stunted growth, bear multiple off-season flowers (most of which fall off), and produce small, irregularly-shaped fruit with a thick, pale peel that remains green at the bottom and tastes bitter.

Label: Detectable marker or reporter molecules, which can be attached to nucleic acids. Typical labels include fluorophores, radioactive isotopes, ligands, chemiluminescent agents, metal sols and colloids, and enzymes. Methods for labeling and guidance in the choice of labels useful for various purposes are discussed, e.g., in Sambrook et al., in Molecular Cloning: A Laboratory Manual, Cold Spring Harbor Laboratory Press (1989) and Ausubel et al., in Current Protocols in Molecular Biology, Greene Publishing Associates and Wiley-Intersciences (1987). A labeled molecule (e.g., a labeled nucleic acid) is a non-naturally occurring molecule.

Pre-symptomatic: Prior o the time when at least certain symptoms are visible or detectable without specialized equipment. In the context of the present disclosure, a pre-symptomatic citrus tree with HLB (a tree that is pre-symptomatic for a HLB infection) is a tree that does not exhibit yellowing of the veins or adjacent tissues, splotchy mottling of the leaf, premature defoliation, dieback of twigs, decay of feeder rootlets or lateral roots, or a decline in vigor attributable to HLB.

Probes & Primers: Nucleic acid probes and primers can be readily prepared based on the nucleic acid molecules provided as indicators of virulence or resistance. It is also appropriate to generate probes and primers based on fragments or portions of these nucleic acid molecules. Also appropriate are probes and primers specific for the reverse complement of these sequences, as well as probes and primers to 5′ or 3′ regions.

A probe comprises an isolated nucleic acid attached to a detectable label or other reporter molecule that is not naturally found connected to the nucleic acid. Typical labels include but are not limited to radioactive isotopes, enzyme substrates, co-factors, ligands, chemiluminescent or fluorescent agents, haptens, and enzymes. More generally, a label is a composition detectable by (for instance) spectroscopic, photochemical, biochemical, immunochemical, or chemical means. Typical labels include fluorescent proteins or protein tags, fluorophores, radioactive isotopes (including for instance ³²P), ligands, biotin, digoxigenin, chemiluminescent agents, electron-dense reagents (such as metal sols and colloids), and enzymes (e.g., for use in an ELISA), haptens, and proteins or peptides (such as epitope tags) for which antisera or monoclonal antibodies are available. Methods for labeling and guidance in the choice of labels useful for various purposes are discussed, e.g., in Sambrook et al., in Molecular Cloning: A Laboratory Manual, Cold Spring Harbor Laboratory Press (1989) and Ausubel et al., in Current Protocols in Molecular Biology, John Wiley & Sons, New York (1998). A label often generates a measurable signal, such as radioactivity, fluorescent light or enzyme activity, which can be used to detect and/or quantitate the amount of labeled molecule.

Primers are short nucleic acid molecules, for instance DNA oligonucleotides 10 nucleotides or more in length. Longer DNA oligonucleotides may be about 15, 20, 25, 30 or 50 nucleotides or more in length. Primers can be annealed to a complementary target DNA strand by nucleic acid hybridization to form a hybrid between the primer and the target DNA strand, and then the primer extended along the target DNA strand by a DNA polymerase enzyme. Primer pairs can be used for amplification of a nucleic acid sequence, e.g., by the polymerase chain reaction (PCR) or other in vitro nucleic-acid amplification methods known in the art.

Methods for preparing and using nucleic acid probes and primers are described, for example, in Sambrook et al. (In Molecular Cloning: A Laboratory Manual, CSHL, New York, 1989), Ausubel et al. (ed.) (In Current Protocols in Molecular Biology, John Wiley & Sons, New York, 1998), and Innis et al. (PCR Protocols, A Guide to Methods and Applications, Academic Press, Inc., San Diego, Calif., 1990). Amplification primer pairs (for instance, for use with polymerase chain reaction amplification) can be derived from a known sequence such as the HgSLP-1 or HgFAR-1 or HgBioB sequences described herein, for example, by using computer programs intended for that purpose such as Primer (Version 0.5, © 1991, Whitehead Institute for Biomedical Research, Cambridge, Mass.).

One of ordinary skill in the art will appreciate that the specificity of a particular probe or primer increases with its length. Thus, for example, a primer comprising 30 consecutive nucleotides of nucleotide sequence from a lemon genome will anneal to a target sequence, such as another homolog of the designated target but from a different citrus, with a higher specificity than a corresponding primer of only 15 nucleotides. Thus, in order to obtain greater specificity, probes and primers can be selected that comprise at least 20, 23, 25, 30, 35, 40, 45, 50 or more consecutive nucleotides of a target-encoding nucleotide sequences.

Quencher: Compound or substance that decreases the fluorescent intensity of a fluorophore.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

III. Introduction

The citrus industry throughout the world is under a serious threat from Huanglongbing (HLB), which has been by far the most devastating disease of citrus. HLB is a vector-borne disease caused by Candidatus Liberibacter, which is transmitted by the Asian citrus psyllid (ACP). There is currently no naturally occurring HLB-resistant citrus cultivar nor is there any cure. Tree removal and aggressive sprays against ACP are the common practices to stop the spread of infection. The situation is further exacerbated by the fact that HLB disease symptoms often take two or more years to appear after the initial Liberibacter exposure. By then the disease may be widespread across the infected grove, thereby making tree removal ineffective for stopping disease spread. Therefore, pre-symptomatic diagnosis of HLB is urgently needed. For this, one needs validated biomarkers that are expressed systemically and early after initial Liberibacter exposure. Disclosed herein is a systems level study performed to discover and validate HLB pre-symptomatic biomarkers.

In the studies described in the examples herein, citrus infection was conducted using infected (Liberibacter+) psyllids in a controlled greenhouse environment. RNA from infected leaves was collected at two early post-inoculation times (8 and 24 weeks). For each of the post-inoculation times, RNA was collected from leaves at three different distances (15, 30 and 60 cm) from the point of Liberibacter inoculation. Genome-wide expression analysis of approximately 44,000 citrus genes was then performed for these greenhouse samples, which identified about 80 citrus genes that are expressed early and systemically upon Liberibacter infection. These pre-symptomatic biomarkers belong to the following citrus innate immune pathways: pathogen-associated molecular pattern (PAMP), PAMP-triggered immunity (PTI), effector-triggered immunity (ETI), and signaling due to the plant hormones salicylic acid (SA), jasmonic acid (JA), and ethylene (ET).

In validation studies, qPCR on FLUIDIGM® Arrays was performed on the RNA greenhouse samples at 0, 2, 4, 8, 16 and 24 weeks post-inoculation and for the same distances (15, 30 and 60 cm) from the point of Liberibacter inoculation. Expression of 80 candidate citrus biomarkers obtained by the discovery process were analyzed. Twenty citrus genes were identified as validated HLB pre-symptomatic biomarkers that show similar expression at most of the time points and distances. These HLB pre-symptomatic biomarkers, and subsets thereof, can be detected on multiple commercial platforms, including qPCR and digital PCR.

These biomarkers enable diagnosis of systemic infection long before the visible symptoms appear in the tree. Once the infected trees are diagnosed in the pre-symptomatic stage, they can be either removed or treated with short-term therapies that are already available (for example, heat and/or chemicals) (Hoffman et al., Phytopathology, 103(1):15-22, 2013; Zhang et al., Phytopathology, 101(9):1097-1103, 2011). The pre-symptomatic diagnosis will be of tremendous utility to any region with citrus groves threatened by HLB, particularly the California, Texas, and Arizona industries that are trying to prevent establishment of HLB, and will also be of value to the Florida citrus industry for monitoring the infection of newly planted trees. The pre-symptomatic diagnosis will slow the disease spread and increase the productive years of citrus groves.

### IV. Overview of Several Embodiments

Described herein is the identification of genes exhibiting altered expression in pre-symptomatic citrus trees following infection by Liberibacter, the causative agent of HLB. Further disclosed are methods for detecting pre-symptomatic infection by Liberibacter in a citrus plant by evaluating expression of at least one of the identified genes.

Provided herein is method of detecting pre-symptomatic infection by Candidatus Liberibacter in a citrus plant. In some embodiments, the method includes measuring expression of at least one, at least two or at least three biomarker genes in a leaf sample obtained from the citrus plant, and detecting pre-symptomatic infection by Candidatus Liberibacter in the citrus plant if expression of the gene(s) is increased compared to a control. In some examples, the at least one, at least two or at least three genes are selected from the orange1.1t04419.1, Cs9g12160.1, Cs2g08750.1, orange1.1t03694.1, orange1.1t04702.1, Cs7g06330.1, Cs5g33540.1, orange1.1t04376.1, orange1.1t03769.1, Cs9g15430.1, Cs5g16850.1, Cs5g16920.1, Cs5g16770.1, Cs5g16780.1, Cs6g04140.1, Cs2g10910.1, Cs5g27580.1, Cs8g01850.1, Cs5g21900.1 and Cs8g01840.1 genes of Citrus sinensis, or a homolog thereof in another citrus plant species (see the Citrus Genome Database at citrusgenomedb.org or the USDA Public Citrus Genome Database at citrus.pw.usda.gov).

Also provided is a method of detecting an increase in expression of at least three biomarker genes (associated with pre-symptomatic infection by Candidatus Liberibacter) in a citrus plant. In some embodiments, the method includes measuring expression of at least three genes in a leaf sample obtained from the citrus plant, wherein the at least three genes are selected from the orange1.1t04419.1, Cs9g12160.1, Cs2g08750.1, orange1.1t03694.1, orange1.1t04702.1, Cs7g06330.1, Cs5g33540.1, orange1.1t04376.1, orange1.1t03769.1, Cs9g15430.1, Cs5g16850.1, Cs5g16920.1, Cs5g16770.1, Cs5g16780.1, Cs6g04140.1, Cs2g10910.1, Cs5g27580.1, Cs8g01850.1, Cs5g21900.1 and Cs8g01840.1 genes of Citrus sinensis, or a homolog thereof in another citrus plant species; and detecting an increase in expression of the at least three genes compared to a control.

In some embodiments of the methods disclosed herein, the method further includes (prior to measuring expression biomarker expression) obtaining the leaf sample from the citrus plant, isolating nucleic acid from the leaf sample, or both.

In some embodiments, measuring expression of the at least one, at least two or at least three genes comprises amplifying nucleic acid isolated from the leaf sample by polymerase chain reaction.

In some embodiments, the nucleic acid is amplified using any one of the pairs of primers listed in Table 2, or any combination of primer pairs listed in Table 2. In some examples, the nucleic acid is amplified using any combination of three pairs of primers listed in Table 2.

In some embodiments, the amplified nucleic acid is detected using a probe comprising the nucleotide sequence of any one of (or any combination of) SEQ ID NOs: 41-60. Probes that are capable of detecting nucleic acid amplified by each primer pair can be identified in Tables 2 and 3 by matching the Assay Code. For example, nucleic acid amplified using the primer pair of SEQ ID NO: 1 and SEQ ID NO: 21 can be detected using the probe of SEQ ID NO: 41. In some examples, the probe is labelled with a fluorophore. In some examples, the probe is labelled with a quencher. In particular examples, the probe is labelled at the 5′ end with a fluorophore and is labelled at the 3′ end with a quencher.

In some embodiments, the method includes measuring at least three citrus genes listed in Table 1, or homologs of one or more thereof. In particular embodiments of the methods, the at least three genes include Cs5g33540.1, Cs6g04140.1 and Cs8g01850.1, or homologs of one or more thereof.

In some embodiments, the method includes measuring expression of at least six citrus genes listed in Table 1, or homologs of one or more thereof. In particular embodiments, the at least six genes includes Cs2g08750.1, orange1.1t03694.1, orange1.1t04702.1, Cs5g33540.1, Cs6g04140.1 and Cs8g01850.1, or homologs of one or more thereof.

In some embodiments, the method includes measuring expression of at least nine citrus genes listed in Table 1, or homologs of one or more thereof. In particular embodiments, the at least nine genes includes Cs9g12160.1, Cs2g08750.1, orange1.1t03694.1, orange1.1t04702.1, Cs5g33540.1, Cs6g04140.1, Cs8g01850.1, Cs5g21900.1 and Cs8g01840.1, or homologs of one or more thereof.

In specific embodiments, the method includes measuring expression of all of the genes listed in Table 1, or homologs thereof.

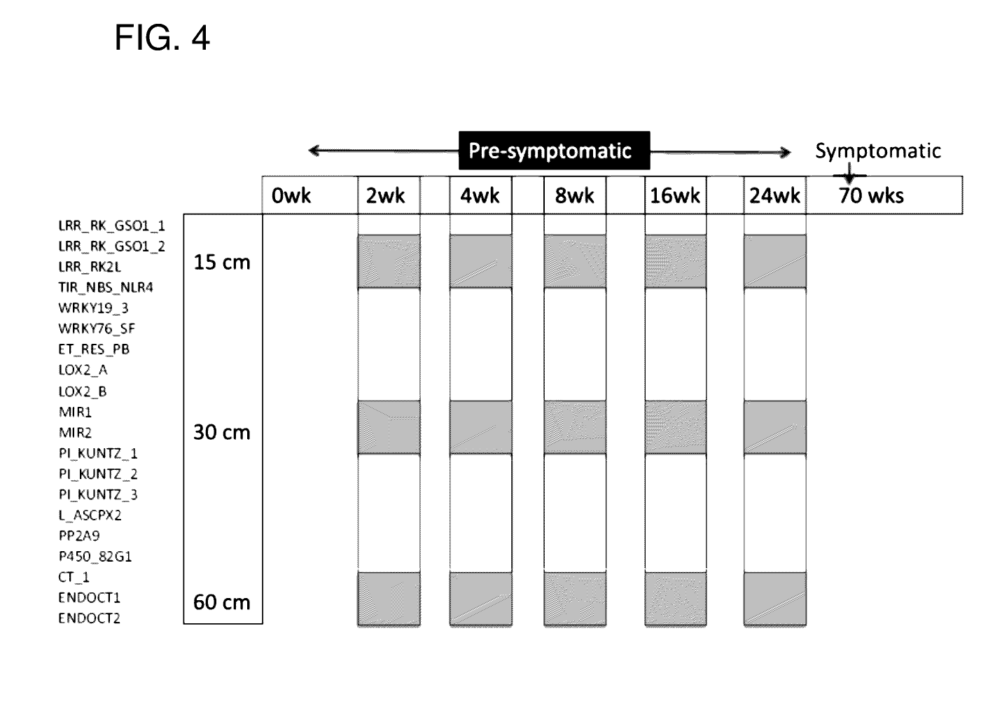

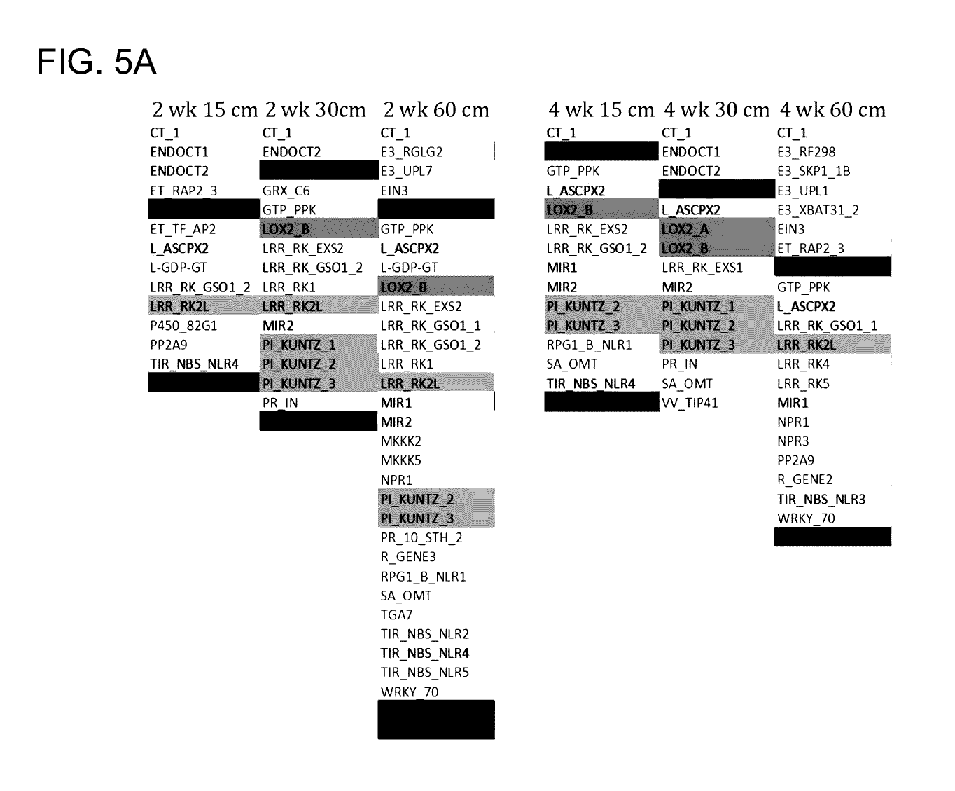

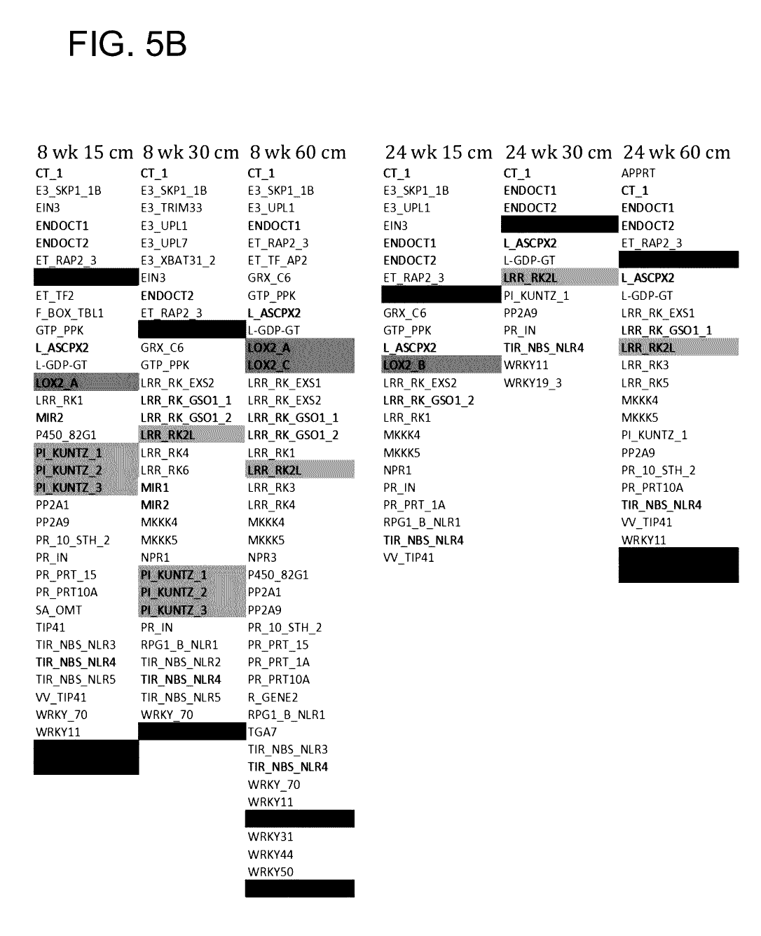

In other embodiments, the at least one, at least two or at least three genes are selected from the genes shown in FIG. 4, FIG. 5A and/or FIG. 5B.

In some embodiments, the method includes measuring expression of the at least one, at least two or at least three genes in a first leaf sample and a second leaf sample, and detecting pre-symptomatic infection by Candidatus Liberibacter asiaticus in the citrus plant if expression of the at least one, at least two or at least three genes is increased in both samples compared to a control. In some examples, the first leaf sample and the second leaf sample are obtained from different locations on the sample plant, such as at least 10 cm, at least 20 cm, at least 30 cm, at least 40 cm, at least 50 cm, at least 60 cm, at least 70 cm, at least 80 cm, at least 90 cm or at least 100 cm apart.

In other embodiments, the method includes measuring expression of the at least one, at least two or at least three genes in a first leaf sample, a second leaf sample and a third leaf sample, and detecting pre-symptomatic infection by Candidatus Liberibacter asiaticus in the citrus plant if expression of the at least one, at least two or at least three genes is increased in at least two of the samples compared to a control. In some examples, the first leaf sample, the second leaf sample and the third leaf sample are obtained from different locations on the sample plant, such as at least 10 cm, at least 20 cm, at least 30 cm, at least 40 cm, at least 50 cm, at least 60 cm, at least 70 cm, at least 80 cm, at least 90 cm or at least 100 cm apart.

In other embodiments of the methods disclosed herein, the leaf sample comprises nucleic acid from at least two leaves or at least three leaves taken from different locations on the same plant. In some examples, at least two leaves or at least three leaves are from locations at least 10 cm, at least 20 cm, at least 30 cm, at least 40 cm, at least 50 cm, at least 60 cm, at least 70 cm, at least 80 cm, at least 90 cm or at least 100 cm apart on the plant.

The citrus plant can be any cultivar of Citrus, including hybrids. In some embodiments, the citrus plant is a tree of the species Citrus sinensis (sweet orange). In other embodiments, the citrus plant is a tree of the species Citrus clementina (clementine), Citrus paradisi (grapefruit), Citrus maxima (pomelo), Citrus limon (lemon), Citrus aurantifolia (lime), Citrus reticulata (Mandarin orange), Citrus tangerina (tangerine), Poncirus trifoliata (trifoliate orange), Citrus medica (citron), or Carrizo citrange (hybrid of C. sinensis and P. trifoliata).

Further provided herein is a kit for detecting pre-symptomatic infection by Candidatus Liberibacter in a citrus plant. In some embodiments, the kit includes at least one primer pair listed in Table 2 and/or at least one probe listed in Table 3. In some examples, the kit includes at least 3, at least 6, at least 9, at least 12 or at least 15 of the primer pairs listed in Table 2, and/or at least 3, at least 6, at least 9, at least 12 or at least 15 of the probes listed in Table 3. In some examples, the probes include a fluorophore. In some examples, the probes include a quencher. In particular examples, the probe is labelled at the 5′ end with a fluorophore and is labelled at the 3′ end with a quencher.

### V. Kits

Kits are provided which contain reagents for determining the (relative) level of expression of one or more of the HLB biomarkers described herein, for instance those shown in Table 1, such as probes or primers specific for at least one of the listed genes or a portion thereof. Alternatively, such probes may be included on an array surface, which is useful in multiplex analysis.

Such kits can be used with the methods described herein to determine whether a sample, such as a tree/leaf sample, contains or is contaminated with Liberibacter or is from a tree that is infected with Liberibacter but pre-symptomatic for HLB. The provided kits may also include written instructions. The instructions can provide calibration curves or charts to compare with the determined (e.g., experimentally measured) values.

Oligonucleotide probes and primers, including those disclosed herein, can be supplied in the form of a kit for use in detection of HLB biomarkers, or more specifically pre-symptomatic infection by Candidatus Liberibacter in a citrus plant, in a sample such as a sample of leaf or other tree tissue. In such a kit, an appropriate amount of one or more of the oligonucleotide primers is provided in one or more containers. The oligonucleotide primers may be provided suspended in an aqueous solution or as a freeze-dried or lyophilized powder, for instance. The container(s) in which the oligonucleotide(s) are supplied can be any conventional container that is capable of holding the supplied form, for instance, microfuge tubes, ampoules, or bottles. In some applications, pairs of primers may be provided in pre-measured single use amounts in individual, typically disposable, tubes or equivalent containers. With such an arrangement, the sample to be tested for (pre-symptomatic) infection by Candidatus Liberibacter, can be added to the individual tubes and amplification carried out directly.

The amount of each oligonucleotide primer supplied in the kit can be any appropriate amount, depending for instance on the market to which the product is directed. For instance, if the kit is adapted for research or clinical use, the amount of each oligonucleotide primer provided would likely be an amount sufficient to prime several PCR amplification reactions. Those of ordinary skill in the art know the amount of oligonucleotide primer that is appropriate for use in a single amplification reaction. General guidelines may for instance be found in Innis et al. (PCR Protocols, A Guide to Methods and Applications, Academic Press, Inc., San Diego, Calif., 1990), Sambrook et al. (In Molecular Cloning: A Laboratory Manual, Cold Spring Harbor, N.Y., 1989), and Ausubel et al. (In Current Protocols in Molecular Biology, Greene Publ. Assoc. and Wiley-Intersciences, 1992).

A kit may include more than two primers, in order to facilitate the in vitro amplification of more than one of the markers listed in Table 1, for instance.

In some embodiments, kits may also include one or more reagents necessary to carry out nucleotide amplification reactions, including, for instance, nucleic acid sample preparation reagents, appropriate buffers (e.g., polymerase buffer), salts (e.g., magnesium chloride), and deoxyribonucleotides (dNTPs).

Kits may in addition include either labeled or unlabeled oligonucleotide probes for use in detection of one or more of the biomarkers listed in Table 1.

The following examples are provided to illustrate certain particular features and/or embodiments. These examples should not be construed to limit the disclosure to the particular features or embodiments described.

### EXAMPLES

### Example 1

### Identification of HLB Pre-Symptomatic Biomarkers

This example describes the identification of 20 biomarkers of the early, pre-symptomatic stages of HLB in citrus trees.

### Methods

Standard methods for the generation of a cDNA fragment library from total RNA were used. Sequencing was performed by loading the denatured double-stranded cDNA on Illumina flow cells (Nagalakshmi et al., Curr Protoc Mol Biol 4.11.1-4.11.13, Jan. 2010).

The gene expression analysis by qPCR (Bustamante et al., Methods Mol Biol, 1110:363-382, 2014) on the FLUIDIGM® Array involved the procedures detailed below.

RNA Isolation and cDNA Generation

RNA was isolated from Hamlin citrus leaves using the Qiagen RNEASY™ Plant Mini kit (Qiagen). The RNA was treated with DNase (Turbo DNA-free, Ambion by Life Technologies). As described by the manufacturer, using TURBO DNA-free, contaminating DNA was digested to levels below the limit of detection by routine PCR. The DNase was then removed rapidly and easily using a method which does not require phenol/chloroform extraction, alcohol precipitation, heating, or the addition of EDTA (Turbo DNA-free, Ambion by Life Technologies product manual). The treated RNA was then analyzed for purity and concentration on a NANODROP™ 1000 spectrophotometer. RNA (75 ng) was converted to cDNA in a 20 μl reaction using High Capacity RNA-to-cDNA (Life Technologies) following the manufacturer's protocol.

### Specific Target Amplification

A total of 94 20× Gene Expression (GE) assays for Citrus sinensis, Candidatus Liberibacter asiaticus and Diaphorina citri were designed and ordered from Life Technologies using the Custom TAQMAN™ Gene Expression assay design tool (available online). GE assays were mixed and diluted with DNA Suspension Buffer (10 mM Tris, pH 8.0, 0.1 mM EDTA) (TEKnova, PN T0221) to prepare a 0.2× pooled assay mixture. 5 μl of TAQMAN™ PreAmp master Mix (2×) (ABI, PN 4391128) was added to 2.5 μl of the 0.2× pooled assay mixture. 2.5 μl of cDNA was then added making a total reaction volume of 10 μl. The reactions were briefly vortexed, centrifuged and placed in a thermal cycler and run using the following conditions: One cycle for 10 minutes at 95° C., followed by 12 cycles at 95° C. for 15 seconds and 60° C. for 4 minutes. After cycling, the reactions were diluted 10-fold by adding 90 μl of DNA suspension Buffer. Reactions were either utilized right away or stored at −80° C. until needed.

### Real Time PCR

The 96.96 Biomark arrays were prepared according to the manufacturer's instructions, except they were run at 2× fluid volumes to prevent evaporation. First, a 96.96 array was loaded with control line fluid and then placed into an Integrated Fluidic Circuit (IFC) controller HX and primed using the Prime (136) script. The 93 TAQMAN™ gene expression assays from ABI were diluted 1:1 with Assay Loading Reagent (Fluidigm, PN 85000736) then 10 μl were loaded into the assay inlets on the primed chip. 10 μl of sample reaction mix was made by adding 5 μl TAQMAN™ Universal PCR Master Mix (2×) (ABI, PN 4304437) to 0.5 μl 20× GE Sample Loading Reagent (Fluidigm, PN85000746), mixing and then combining with 4.5 μl of the STA pre-amplified cDNA. 10 μl of sample mix was loaded into the sample inlets of the array. The chip was then loaded into the IFC controller HX and run using the Load Mix (136×) script to load and mix the assays into the chip. The chip was then loaded and run on the BioMark Real time PCR system. The standard GE thermal protocol was used. This consisted of a Thermal Mix phase—1 cycle 50° C. for 2 minutes, 1 cycle 70° C. for 30 minutes and 1 cycle 25° C. for 10 minutes. A Uracil-N-glycosylase (UNG) activation cycle—50° C. for 2 minutes and a hot start for Taq polymerase cycle—95° C. were run prior to 40 rounds of PCR cycling at 95° C. denaturing for 15 seconds followed by 60° C. anneal and data capture for 1 minute.

### Background

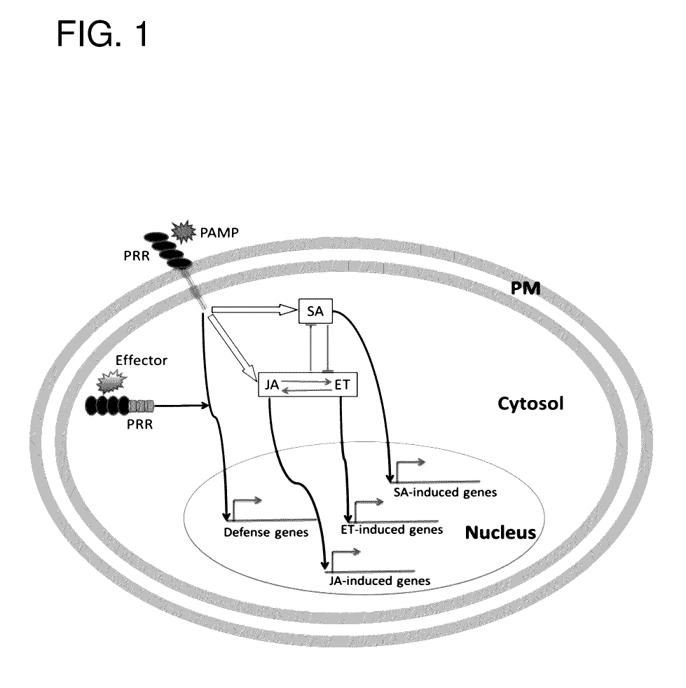

In this study, the gene expression pattern of citrus during the early stages of infection was evaluated to identify pre-symptomatic biomarkers. Several such studies have been done on the model plant Arabidopsis thaliana infected with both bacterial and fungal pathogens (Macho and Zipfel, Mol Cell, 54(2):263-72, 2014; Steinbrenner et al., Cold Spring Harb Symp Quant Biol, 77:249-257, 2012; Schwessinger and Ronald, Annu Rev Plant Biol, 63:451-482, 2012). These studies, as shown in FIG. 1, revealed that multiple processes are involved during the early stages infection. Pathogen-associated molecular patterns (PAMPs) are recognized by PAMP recognition receptors (PRR) on the plasma membrane and PAMP-triggered immunity (PTI) causes the induction of defense genes. In addition, PRRs are also present in the cytosol and they recognize pathogen effectors and cause effector-triggered immunity (ETI), which merges with the PTI signaling. The plant defense is further elaborated by the recruitment of small hormones upon pathogen attack. These include salicylic acid (SA), jasmonic acid (JA), and ethylene (ET), which act as global regulators of plant defense signaling. SA appears to play a central role as a signaling molecule involved in both local and systemic defense (Reymond and Farmer, Curr Opin Plant Biol, 1(5):404-411, 1998). JA and ET seem to cooperate with each other (Kazan et al., Plant Physiol, 146(4):1459-1468, 2008; Broekaert et al., Annu Rev Phytopathol, 44:393-416, 2006; Kunkel and Brooks, Curr Opin Plant Biol, 5(4):325-331, 2002). Methyl-SA and methyl-JA are volatile, like ET, and capable of inducing air-borne effect at a distance (Yi et al., Plant Physiol, 151(4):2152-2161, 2009). SA may exert inhibitory effects on JA and ET and vice versa. Finally, the stress (for example, the formation of reactive oxygen and nitrogen species) caused by the pathogen attack tends to influence both the immune (PTI and ETI) and plant hormone (SA, JA, and ET) pathways (Scheler et al., Curr Opin Plant Biol, 16(4):534-539, 2013).

As shown in FIG. 1, the plant immune and hormone pathways exert both stimulatory and inhibitory effects on each other. The crosstalk among various pathways may be altered by pathogen mimicry, which involves production of plant hormone mimics by the pathogen. For example, Pseudomonas syringe produces a JA analog (called Coronatine), which may induce JA-responsive genes in plants and also inhibit SA-responsive pathways to the detriment of the plant. In addition, several pseudomonads produce SA analogs and many bacteria produce ET. It is not clear how production of SA analogs and ET offer bacteria an advantage for countering plant defense. Nonetheless since different pathogens contain different JA/SA analogs and different levels of ET, the net strength of the stimulatory and inhibitory effects may determine which genes are expressed at what level and at what time by a given pathogen.

The immune, stress, and hormone pathways induced by bacterial pathogens have also been identified in tomato, tobacco, and rice (Nandety et al., Plant Physiol, 162(3):1459-1472, 2013; Bhattarai et al., Plant J, 63(2):229-240, 2010; Newman et al., Front Plant Sci, 4:139, 2013). Normally these pathways are induced to block infection. But pathogens have evolved strategies to subvert these pathways to establish a niche in the host.

The experimental design for the gene expression disclosed herein uses Liberibacter-carrying (hot) psyllid for inoculation in a single branch, rather than exposing entire canopies or using graft inoculation. In addition, RNAs for analyzing expression are collected very early after infection at a distance from the actual inoculation sites. A range of post-inoculation time-points within 0-24 weeks were used to capture the biomarkers for the pre-symptomatic stage and also from leaves located at different distances from the point of inoculation to capture truly systemic biomarkers.

### Study Design

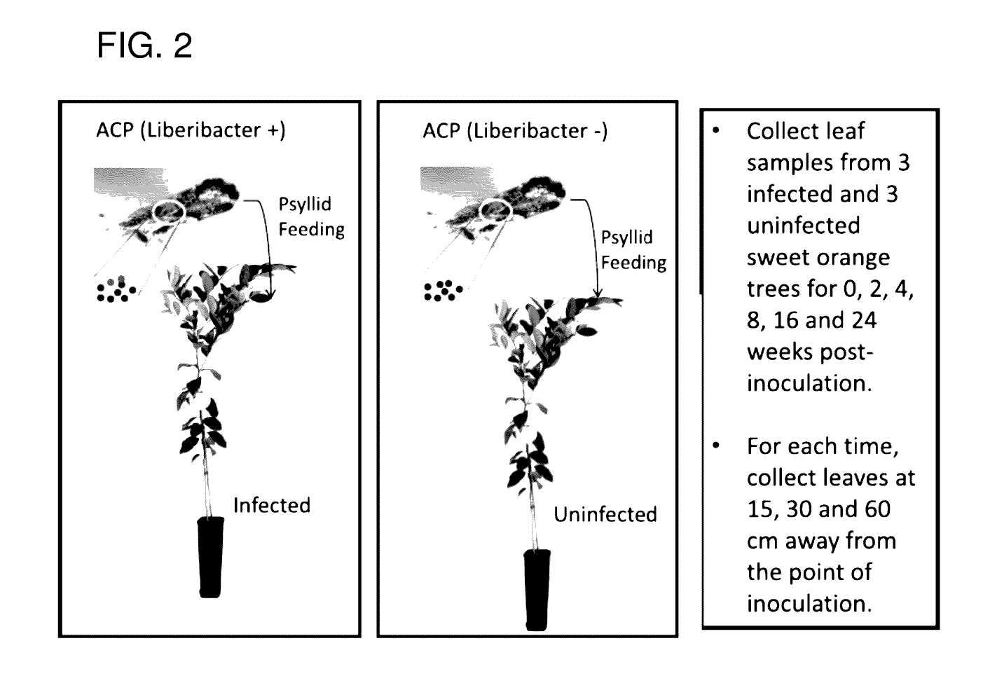

The infection study was carried out using Hamlin sweet orange trees on Carrizo rootstock in the controlled greenhouse environment. The study was divided into two groups. In one group, three citrus trees were infected with Liberibacter+ ACP whereas the other group was exposed to feeding by Liberibacter− ACP. Each tree contained a cage at the end of a branch and the cage was filled with 75 Liberibacter (+or −) ACP (FIG. 2A). RNA from leaf samples from each group was collected at 0, 2, 4, 8, 12 and 24 weeks post-inoculation. For each time-point, RNA was sampled from leaves 15, 30 and 60 cm from the point of inoculation. Typically symptoms appear about a year or more after initial Liberibacter exposure. In the present study, the ACP carried a low Liberibacter titer as would be expected when the disease first enters an area, and the infected trees developed leaf mottling (characteristic of HLB) after 2 years (FIG. 2B). Therefore, expression analysis using RNA samples from 0-24 weeks post-inoculation proved to be suitable for analysis of pre-symptomatic effects. Also, testing RNA extracted from leaves 3 separate distances from the point of inoculation permitted analysis of local and systemic effects during the early stages of infection. Finally, differential gene expression analysis of citrus exposed to Liberibacter (+and −) ACP allowed for the capture of the genes specifically induced by Liberibacter, not by stress caused during ACP feeding or due to exposure to the natural ACP microbiome.

Genome-wide expression (RNA-seq) data were collected using the RNA samples from infected and uninfected trees (FIG. 2C) for 8 and 24 weeks post-inoculation and for distances 15, 30 and 60 cm away from the point of inoculation. Protocols as described in Nagalakshmi et al. (Curr Protoc Mol Biol 4.11.1-4.11.13, January 2010) were followed for RNA-seq. Differential (Infected vs. Uninfected) expression of 44,000 citrus genes was analyzed. Genes significantly altered in expression were identified by imposing two filters: (i) at least 10 counts per million for a gene in the infected or uninfected sample and (ii) ±2-fold or greater change in expression at 8 or 24 weeks post-inoculation at one or more of the three sampled distances. This analysis identified 80 citrus genes as candidate biomarkers for HLB pre-symptomatic diagnosis.

### Discovery Process

Of the 80 candidate biomarker genes, most exhibited up-regulation at both 8 and 24 weeks post-inoculation. Many of these genes were expressed at two sites from the point of inoculation for a given post-inoculation time. Disease resistance and pathogenesis-related genes showed systemic expression (expressed at two distances). Leucine-rich repeat (LRR) receptor and NLR genes showed higher level of expression upon infection only at 60 cm away from the point of inoculation. A few genes (such as NPR1, one MAPKK2 and COL1) were down-regulated systemically upon infection. These genes were expressed at a high level in uninfected citrus trees and therefore, their down-regulation can be reliably monitored. A significant fraction of the 80 genes were up-regulated at 15 cm (close to the point of inoculation).

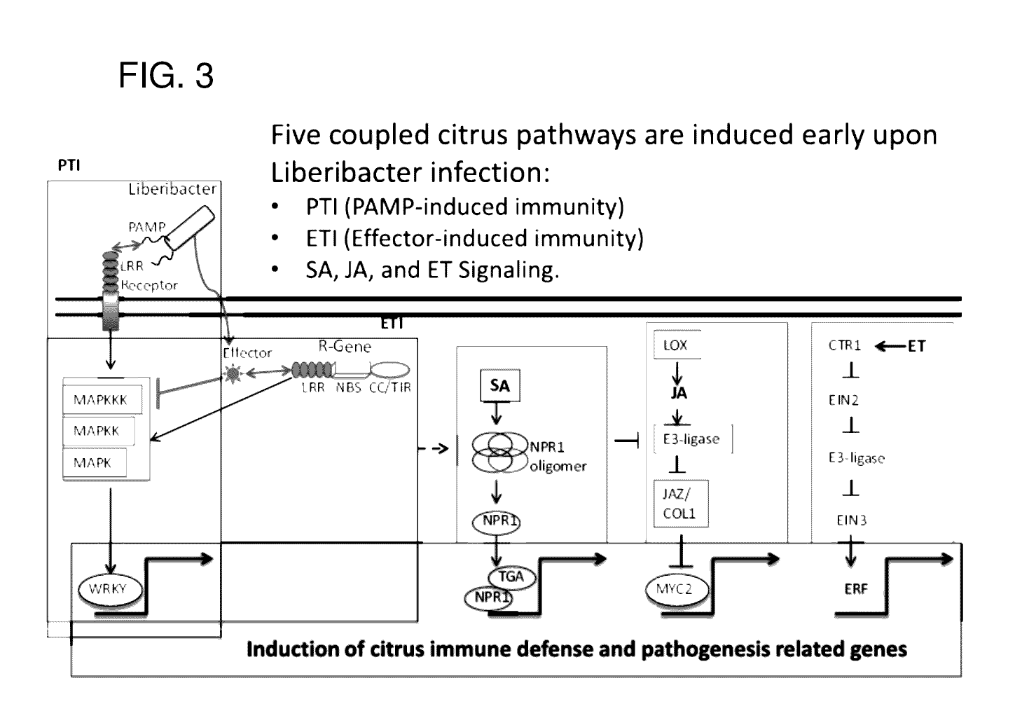

Genes belonging to the innate immune defense system were significantly altered in their expression during the early stages of infection. This defense mainly consisted of PTI, ETI, and SA/JA/ET signaling pathways, the net effect of which determined the status of infection. These pathways are coupled (FIG. 3). The PTI pathway includes the leucine-rich repeat (LRR) receptor family that may recognize extracellular flagellin, chitin, or elongation factor Tu, which, in turn, leads to downstream signaling via mitogen-activated protein (MAP) kinase family and WRKY family transcription factors. Differentially expressed genes in this study included 100, 40, and 30 genes belonging, respectively, to LRR receptor, MAP kinase, and WRKY families. The LRR receptors were down-regulated near the site of inoculation, probably due to inhibitory effects by intracellular Liberibacter effectors (FIG. 3), and were up-regulated away from the site of inoculation. About 30 resistance (R) genes were also induced likely to counter the Liberibacter effectors. However, many of the R-genes were down-regulated near the site of inoculation but up-regulated away (i.e., at 60 cm) from the point of inoculation. In addition to PTI and ETI, SA/JA/ET signaling pathways were also induced. SA signaling is triggered subsequent to pathogen infection as a consequence of PTI/ETI, reactive oxygen and nitrogen species, and pathogen-induced stress. SA O-methyltransferase, (SA-OMT), nonexpressor of pathogenesis-related genes 1,3 (NPR1, NPR3), Gutaredoxin C-6, and TGA genes in the SA-signaling pathway were significantly altered upon infection. SA O-methyltransferase converts SA into volatile O-methyl SA, which may offer SA-induced pathogen resistance at a distance in the same plant or in the neighboring plants. This gene was up-regulated both at 8 and 24 weeks of post-inoculation.

The NPR1 gene however, was down-regulated. Monomeric NPR1 and the transcription factor TGA are critical for the expression of SA-induced genes (FIG. 3). TGA genes were down-regulated close to the point of inoculation and up-regulated away from the point of inoculation. Both Glutaredoxin C-6 (an activator of NPR1) and NPR3 (an inhibitor of NPR1) were up-regulated upon infection. Glutaredoxin C-6 converts S-S bridged inactive NPR1 multimers into active monomer whereas NPR3 (SA-receptor and a paralog of NPR1) in association with E3-ligase directs the degradation of NPR1 by the proteosome. Therefore, this data showed that SA-signaling was inhibited close to the point of inoculation (Reymond and Farmer, Curr Opin Plant Biol, 1(5):404-411, 1998; Kazan et al., Plant Physiol, 146(4):1459-1468, 2008). In the JA-signaling pathway, lysil oxidase (LOX), S-Phase Kinase-Associated (SKP1, an E3-ligase), jasmonate ZIM-domain/CONSTANS-like 1 (JAZ/COL1), and the basic helix-loop-helix transcription factor MYC2 were significantly altered upon infection. LOX, involved in JA synthesis, was up-regulated upon infection both at 8 and 24 weeks of post-inoculation and so were SKP1 and COL1, which are inhibitors of MYC2 (FIG. 3). MYC2 is critical for the expression of the JA-induced genes. Thus, these data showed JA-signaling was suppressed during the early stage of Liberibacter infection. On the contrary, the ET-signaling was found to be activated upon Liberibacter infection, i.e., the constitutive triple response 1 (CTR1) gene was down-regulated, whereas ethylene insensitive 3 (EIN3) and the transcription factor ERF were both up-regulated. Critical to PTI, ETI, SA, JA, and ET signaling are the E3-ligase family of genes, some of which were up-regulated, whereas some others were down-regulated upon infection. The expression pattern in the E3-ligase gene family clearly presents signatures of early stage Liberibacter infection. Finally, the outputs from PTI, ETI, SA, JA, and ET are the expression of disease resistance and pathogenesis related genes, which include citrus protease inhibitors, chitinases, phloem protein 2A/B, cytochrome oxidase P450, peroxidases, PR-genes 1/10, etc. Many of these genes were up-regulated both at 8 and 24 weeks of post-infection and systemically expressed (see FIGS. 5A-5B).

### Validation Process

The 80 candidate discovered biomarkers were subjected to further analysis using greenhouse samples. As shown in FIG. 4, samples for 2, 4, 8, 16 and 24 weeks of post-inoculation for all three distances (15, 30, and 60 cm) from the site of inoculation were analyzed. The period of 0-24 weeks post-inoculation define the pre-symptomatic stage. The visual symptoms were verified after 70 weeks. Therefore, samples for 70 weeks post-inoculation were also analyzed as controls for symptomatic trees.

As shown in the left-most panel, a subset of 20 genes (out of approximately 80 candidate genes) showed similar expression at most early time points and distances of the greenhouse samples. Therefore, these 20 genes were chosen as the validated HLB pre-symptomatic biomarkers. Table 1 provides a list of the 20 validated biomarkers, identified by assay code, gene ID and gene name. Sequences for the 20 genes from a variety of citrus species, including Citrus sinensis, are publically available through the Citrus Genome Database (citrusgenomedb.org) or the USDA Public Citrus Genome Database (citrus.pw.usda.gov), which are maintained online (see also Talon and Gmitter, Int J Plant Genomics 2008:528361, 2008). Table 2 provides the sequences of the forward and reverse primers used to amplify the 20 biomarker genes and Table 3 provides probe sequences for each biomarker gene.

TABLE 1
Validated Biomarkers of Pre-Symptomatic HLB

| Assay Code | Gene ID | Gene Name |
|---|---|---|
| LRR_RK_GSO1_1 | orange1.1t04419.1 | LRR receptor-like serine/threonine-protein kinase GSO1 |
| | | (LRR-RK-GSO1-1) |
| LRR_RK_GSO1_2 | Cs9g12160.1 | LRR receptor-like serine/threonine-protein kinase GSO1 |
| | | (LRR-RK-GSO1-2) |
| LRR_RK2L | Cs2g08750.1 | Probable LRR receptor-like serine/threonine-protein |
| | | kinase At3g47570 (LRR-RK2) |
| TIR_NBS_NLR4 | orange1.1t03694.1 | TIR-NBS-LRR-TIR type disease resistance protein |
| | | (Fragment) (TIR-NBS-NLR4) |
| WRKY19_3 | orange1.1t04702.1 | Probable WRKY transcription factor 19 (WRKY19-3) |
| WRKY76_SF | Cs7g06330.1 | WRKY76-superfamily of TFs having WRKY and zinc |
| | | finger domains (WRKY76-SF) |
| ET_RES_PB | Cs5g33540.1 | Ethylene-responsive element binding protein (Fragment) |
| | | (ET-RES-PB) |
| LOX2_A | orange1.1t04376.1 | Linoleate 13S-lipoxygenase 2-1% 2C chloroplastic |
| | | (LOX2-A) |
| LOX2_B | orange1.1t03769.1 | Linoleate 13S-lipoxygenase 2-1% 2C chloroplastic |
| | | (LOX2-B) |
| MIR1 | Cs9g15430.1 | Miraculin-1 |
| MIR2 | Cs5g16850.1 | Miraculin-2 |
| PI_KUNTZ_1 | Cs5g16920.1 | Kunitz-type protease inhibitors (PI-Kuntz-1) |
| PI_KUNTZ_2 | Cs5g16770.1 | Kunitz-type protease inhibitors (PI-Kuntz-2) |
| PI_KUNTZ_3 | Cs5g16780.1 | Kunitz-type protease inhibitors (PI-Kuntz-3) |
| L_ASCPX2 | Cs6g04140.1 | L-ascorbate peroxidase 2 (L-ASC-Peroxydase) |
| PP2A9 | Cs2g10910.1 | PR Protein (PP2A9) |
| P450_82G1 | Cs5g27580.1 | Cytochrome P450 82G1 |
| CT_1 | Cs8g01850.1 | Chitinase (CT1) |
| ENDOCT1 | Cs5g21900.1 | Endochitinase PR4 (EndoCT1) |
| ENDOCT2 | Cs8g01840.1 | Endochitinase (EndoCT2) |

TABLE 2
Primer Sequences

| | Forward Primer | SEQ ID | Reverse Primer | SEQ ID |
|---|---|---|---|---|
| Assay Code | Sequence | NO: | Sequence | NO: |
| LRR_RK_GSO1_1 | AGGCAATCTTTCTCAA | 1 | TGGTCAAGTTGCTGATCT | 21 |
| | TCTATTGAAGAGTTT | | CTTTAGG | |
| LRR_RK_GSO1_2 | ATTCACACAGATGGTT | 2 | CATTCATTGAAATGTTGA | 22 |
| | AAGATTCTTGGA | | AGGATATTAAACTTGGT | |
| LRR_RK2L | TGGAGGCCTAACTAAT | 3 | GGATTTCAAGCTTATCAA | 23 |
| | TTACAATATCTCTTCT | | ATCACCAAATGA | |
| TIR_NBS_NLR4 | TCAAATGGCATGGATA | 4 | ATGCGACTATTACACAGG | 24 |
| | CCCGTTT | | TTCAACTT | |
| WRKY19_3 | TGGGATTGTGTGAAGC | 5 | CGTGGGATTTGGAATTTT | 25 |
| | ATTTTAGTAAGT | | GGCAAT | |
| WRKY76_SF | GGCTTCTTCTGGATGTC | 6 | TGTTCTCCTTCATAAGTT | 26 |
| | CTGTAAA | | GCAACGA | |
| ET_RES_PB | CCCAACGAAGAGGACG | 7 | CTGGGAGTGGGATTATG | 27 |
| | TCTT | | ATTATGCT | |
| LOX2_A | GCACTCCCCAAAGACC | 8 | TCAAGCCATGTGGAGCA | 28 |
| | TAATTAGC | | CTT | |
| LOX2_B | TCGAACACACGACCTT | 9 | GGTCTCAACGTGTTATCT | 29 |
| | GTATGG | | GGAGTTA | |
| MIR1 | TTGAGGGAACTCCAGT | 10 | CCCTAATCACCTTCCCTT | 30 |
| | TACTAAGGA | | TCTTGTT | |
| MIR2 | GCTGCATCAGGCAAAT | 11 | TCCAATTTTCTCAAGCTT | 31 |
| | GGTTTATA | | AAACCAATTTAGC | |
| PI_KUNTZ_1 | ACCAGGTGCATACAAA | 12 | CGCCATCCTCAAACGAA | 32 |
| | ATTGTTCATTG | | AAGC | |
| PI_KUNTZ_2 | CTATCTTGTCCTAGAC | 13 | ACGTCGAACGCCATCCTT | 33 |
| | GCTGCAA | | AG | |
| PI_KUNTZ_3 | CAAGAGTTTCTTCTCTT | 14 | TGCTTTCCTAATGAAGCG | 34 |
| | CAGCAGTGT | | TTATCGT | |
| L_ASCPX2 | CACATGGGTCTGAGTG | 15 | CTTGTGGCACCTACCCAA | 35 |
| | ATAAGGAT | | TGT | |
| PP2A9 | CAGCTCTATTTTGGACT | 16 | ACTTCTCTGATGAATGCA | 36 |
| | GTATGAGGTA | | TGGTGAA | |
| P450_82G1 | GGCAACTGGCTTGAAG | 17 | AGCAACACGTCCATGAA | 37 |
| | AACAT | | GTCA | |
| CT_1 | CCGAGGTCCAATTCAA | 18 | GCAAGTAGGTCTGGATTG | 38 |
| | CTCACTT | | TTCAACA | |
| ENDOCT1 | CAATATCCATGCAATC | 19 | CAGGTCCGTAGTTGAAAT | 39 |
| | CGAGCAAA | | TCCAAGA | |
| ENDOCT2 | CGGTGGAATTGAATGT | 20 | GGTAAAGAACCCAATAC | 40 |
| | GGCTAAG | | GGTTACGT | |

TABLE 3
FAM/MGB-Labelled Probes

| Assay Code | Probe Sequence | SEQ ID NO: |
|---|---|---|
| LRR_RK_GSO1_1 | AATGCCGCCACTAATG | 41 |
| LRR_RK_GSO1_2 | TCCAAGGCCACATTCC | 42 |
| LRR_RK2L | CAAGGCTCGATTCCTG | 43 |
| TIR_NBS_NLR4 | TTTGCCCGTGAGTTTC | 44 |
| WRKY19_3 | CTGCCTGCCATAAGCT | 45 |
| WRKY76_SF | CCTCCATGCATCTTTG | 46 |
| ET_RES_PB | CTGCCGCCCATGCTT | 47 |
| LOX2_A | CAACAGCCAATCCC | 48 |
| LOX2_B | CCCGCACTGTATTCTT | 49 |
| MIR1 | ACCCAAACACGAACCC | 50 |
| MIR2 | CTGGAGCTGAAACTTT | 51 |
| PI_KUNTZ_1 | TCTTGCGTCAAACTC | 52 |
| PI_KUNTZ_2 | TTGAAACGCCAATTTT | 53 |
| PI_KUNTZ_3 | CCACAACGAATCTTTG | 54 |
| L_ASCPX2 | ATGACCGCCGGATAAA | 55 |
| PP2A9 | CTCCCTTCCACTTTCC | 56 |
| P450_82G1 | TCACCCTGTAATTTTC | 57 |
| CT_1 | CCCCAAGGCTTCTCC | 58 |
| ENDOCT1 | CCCGGCCATAGTAACC | 59 |
| ENDOCT2 | CAGCGGCATTCCCAC | 60 |

### Example 2

### Detection of Biomarkers in Citrus Trees

This Example provides an exemplary system for detecting changes (e.g., increases) in the level of one or more biomarkers indicative of pre-symptomatic HLB in samples from citrus trees.

Leaf (or other plant/tree tissue) samples are taken, for instance, from a grove in which citrus trees may have been exposed to Liberibacter. By way of example, the leaf sample may be a combination of multiple leaves taken from different locations of the tree. In other examples, multiple leaves (such as two or three leaves) are taken from different locations on the tree and processed as separate samples. In some instances, a single leaf sample is obtained from a tree to be tested. RNA is then isolated from the leaf sample(s), reverse transcribed and amplified by PCR using primers specific for the HLB biomarkers disclosed herein. Exemplary primers and probes are provided in Tables 2 and 3.

The level of biomarker expression in the leaf sample is compared to a control sample, such as an uninfected leaf sample, or a historical standard/standard value. An increase in expression of one or multiple biomarkers listed in Table 1 (and/or FIG. 4 and/or FIGS. 5A-5B) indicates the citrus tree is infected with Liberibacter.

The results of such a biomarker analysis can be used, for instance, in decisions regarding how to treat the field from which the sample(s) were obtained. By way of example, in heavily infected fields (that is, fields with a relatively high level of trees with HLB), the field might be aggressively treated to eradicate Liberibacter. Alternatively, some or all of the trees that are found to be infected with Liberibacter may be destroyed, possibly in combination with other treatments of the remainder of the field or surrounding area.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A method of detecting pre-symptomatic infection by Candidatus Liberibacter asiaticus in a citrus plant, comprising:
measuring expression of at least three genes in a leaf sample obtained from the citrus plant, wherein the at least three genes are selected from the orange1.1t04419.1, Cs9g12160.1, Cs2g08750.1, orange1.1t03694.1, orange1.1t04702.1, Cs7g06330.1, Cs5g33540.1, orange1.1t04376.1, orange1.1t03769.1, Cs9g15430.1, Cs5g16850.1, Cs5g16920.1, Cs5g16770.1, Cs5g16780.1, Cs6g04140.1, Cs2g10910.1, Cs5g27580.1, Cs8g01850.1, Cs5g21900.1 and Cs8g01840.1 genes of Citrus sinensis, or a homolog thereof in another citrus plant species; and
detecting pre-symptomatic infection by Candidatus Liberibacter asiaticus in the citrus plant if expression of the at least three genes is increased compared to a control.

2. The method of claim 1, further comprising obtaining the leaf sample from the citrus plant and isolating nucleic acid from the leaf sample prior to measuring expression.

3. The method of claim 1, wherein measuring expression of the at least three genes comprises amplifying nucleic acid isolated from the leaf sample by polymerase chain reaction.

4. The method of claim 3, wherein the nucleic acid is amplified using any one of the following pairs of primers:
primers comprising the nucleotide sequence of SEQ ID NO: 1 and SEQ ID NO: 21;
primers comprising the nucleotide sequence of SEQ ID NO: 2 and SEQ ID NO: 22;
primers comprising the nucleotide sequence of SEQ ID NO: 3 and SEQ ID NO: 23;
primers comprising the nucleotide sequence of SEQ ID NO: 4 and SEQ ID NO: 24;
primers comprising the nucleotide sequence of SEQ ID NO: 5 and SEQ ID NO: 25;
primers comprising the nucleotide sequence of SEQ ID NO: 6 and SEQ ID NO: 26;
primers comprising the nucleotide sequence of SEQ ID NO: 7 and SEQ ID NO: 27;
primers comprising the nucleotide sequence of SEQ ID NO: 8 and SEQ ID NO: 28;
primers comprising the nucleotide sequence of SEQ ID NO: 9 and SEQ ID NO: 29;
primers comprising the nucleotide sequence of SEQ ID NO: 10 and SEQ ID NO: 30;
primers comprising the nucleotide sequence of SEQ ID NO: 11 and SEQ ID NO: 31;
primers comprising the nucleotide sequence of SEQ ID NO: 12 and SEQ ID NO: 32;
primers comprising the nucleotide sequence of SEQ ID NO: 13 and SEQ ID NO: 33;
primers comprising the nucleotide sequence of SEQ ID NO: 14 and SEQ ID NO: 34;
primers comprising the nucleotide sequence of SEQ ID NO: 15 and SEQ ID NO: 35;
primers comprising the nucleotide sequence of SEQ ID NO: 16 and SEQ ID NO: 36;
primers comprising the nucleotide sequence of SEQ ID NO: 17 and SEQ ID NO: 37;
primers comprising the nucleotide sequence of SEQ ID NO: 18 and SEQ ID NO: 38;
primers comprising the nucleotide sequence of SEQ ID NO: 19 and SEQ ID NO: 39; and/or
primers comprising the nucleotide sequence of SEQ ID NO: 20 and SEQ ID NO: 40.

5. The method of claim 3, wherein the amplified nucleic acid is detected using a probe comprising the nucleotide sequence of any one of SEQ ID NOs: 41-60.

6. The method of claim 5, wherein the probe is labelled with a fluorophore.

7. The method of claim 5, wherein the probe is labelled with a quencher.

8. The method of claim 1, wherein the at least three genes comprises Cs5g33540.1, Cs6g04140.1 and Cs8g01850.1, or homologs of one or more thereof.

9. The method of claim 1, comprising measuring expression of at least six genes in a leaf sample obtained from the citrus plant, wherein the at least six genes are selected from the orange1.1t04419.1, Cs9g12160.1, Cs2g08750.1, orange1.1t03694.1, orange1.1t04702.1, Cs7g06330.1, Cs5g33540.1, orange1.1t04376.1, orange1.1t03769.1, Cs9g15430.1, Cs5g16850.1, Cs5g16920.1, Cs5g16770.1, Cs5g16780.1, Cs6g04140.1, Cs2g10910.1, Cs5g27580.1, Cs8g01850.1, Cs5g21900.1 and Cs8g01840.1 genes of Citrus sinensis, or a homolog thereof in another citrus species.

10. The method of claim 9, wherein the at least six genes comprises Cs2g08750.1, orange1.1t03694.1, orange1.1t04702.1, Cs5g33540.1, Cs6g04140.1 and Cs8g01850.1, or homologs of one or more thereof.

11. The method of claim 1, comprising measuring expression of at least nine genes in a leaf sample obtained from the citrus plant, wherein the at least nine genes are selected from the orange1.1t04419.1, Cs9g12160.1, Cs2g08750.1, orange1.1t03694.1, orange1.1t04702.1, Cs7g06330.1, Cs5g33540.1, orange1.1t04376.1, orange1.1t03769.1, Cs9g15430.1, Cs5g16850.1, Cs5g16920.1, Cs5g16770.1, Cs5g16780.1, Cs6g04140.1, Cs2g10910.1, Cs5g27580.1, Cs8g01850.1, Cs5g21900.1 and Cs8g01840.1 genes of Citrus sinensis, or a homolog thereof.

12. The method of claim 11, wherein the at least nine genes comprises Cs9g12160.1, Cs2g08750.1, orange1.1t03694.1, orange1.1t04702.1, Cs5g33540.1, Cs6g04140.1, Cs8g01850.1, Cs5g21900.1 and Cs8g01840.1, or homologs of one or more thereof.

13. The method of claim 1, comprising measuring expression of orange1.1t04419.1, Cs9g12160.1, Cs2g08750.1, orange1.1t03694.1, orange1.1t04702.1, Cs7g06330.1, Cs5g33540.1, orange1.1t04376.1, orange1.1t03769.1, Cs9g15430.1, Cs5g16850.1, Cs5g16920.1, Cs5g16770.1, Cs5g16780.1, Cs6g04140.1, Cs2g10910.1, Cs5g27580.1, Cs8g01850.1, Cs5g21900.1 and Cs8g01840.1.

14. The method of claim 1, comprising measuring expression of the at least three genes in a first leaf sample and a second leaf sample, and detecting pre-symptomatic infection by Candidatus Liberibacter asiaticus in the citrus plant if expression of the at least three genes is increased in both samples compared to a control.

15. The method of claim 14, comprising measuring expression of the at least three genes in a first leaf sample, a second leaf sample and a third leaf sample, and detecting pre-symptomatic infection by Candidatus Liberibacter asiaticus in the citrus plant if expression of the at least three genes is increased in at least two of the samples compared to a control.

16. The method of claim 1, wherein the leaf sample comprises nucleic acid from at least two leaves or at least three leaves taken from different locations on the same plant.

17. The method of claim 1, wherein the citrus plant is a tree of the species Citrus sinensis.

18. The method of claim 1, wherein the citrus plant is a tree of the species Citrus sinensis, Citrus clementine or Citrus Carrizo.

19. A kit comprising at least one pair of primers selected from:
primers comprising the nucleotide sequence of SEQ ID NO: 1 and SEQ ID NO: 21;
primers comprising the nucleotide sequence of SEQ ID NO: 2 and SEQ ID NO: 22;
primers comprising the nucleotide sequence of SEQ ID NO: 3 and SEQ ID NO: 23;
primers comprising the nucleotide sequence of SEQ ID NO: 4 and SEQ ID NO: 24;
primers comprising the nucleotide sequence of SEQ ID NO: 5 and SEQ ID NO: 25;
primers comprising the nucleotide sequence of SEQ ID NO: 6 and SEQ ID NO: 26;
primers comprising the nucleotide sequence of SEQ ID NO: 7 and SEQ ID NO: 27;
primers comprising the nucleotide sequence of SEQ ID NO: 8 and SEQ ID NO: 28;
primers comprising the nucleotide sequence of SEQ ID NO: 9 and SEQ ID NO: 29;
primers comprising the nucleotide sequence of SEQ ID NO: 10 and SEQ ID NO: 30;
primers comprising the nucleotide sequence of SEQ ID NO: 11 and SEQ ID NO: 31;
primers comprising the nucleotide sequence of SEQ ID NO: 12 and SEQ ID NO: 32;
primers comprising the nucleotide sequence of SEQ ID NO: 13 and SEQ ID NO: 33;
primers comprising the nucleotide sequence of SEQ ID NO: 14 and SEQ ID NO: 34;
primers comprising the nucleotide sequence of SEQ ID NO: 15 and SEQ ID NO: 35;
primers comprising the nucleotide sequence of SEQ ID NO: 16 and SEQ ID NO: 36;
primers comprising the nucleotide sequence of SEQ ID NO: 17 and SEQ ID NO: 37;
primers comprising the nucleotide sequence of SEQ ID NO: 18 and SEQ ID NO: 38;
primers comprising the nucleotide sequence of SEQ ID NO: 19 and SEQ ID NO: 39; and
primers comprising the nucleotide sequence of SEQ ID NO: 20 and SEQ ID NO: 40.

20. The kit of claim 19, further comprising at least one probe comprising the nucleotide sequence of any one of SEQ ID NOs: 41-60.
